(19) United States
(12) Patent Application Publication
(10) Pub. No.: US 2010/0197785 A1
(43) Pub. Date: Aug. 5, 2010

(54) OMEGA-3 FATTY ACID FORTIFIED COMPOSITION

(75) Inventor: Harald Breivik, Porsgrunn (NO)

Correspondence Address: VOLPE AND KOENIG, P.C., UNITED PLAZA, SUITE 1600, 30 SOUTH 17TH STREET, PHILADELPHIA, PA, 19103, US

(73) Assignee: EPAX AS, Ålesund (NO)

(21) Appl. No.: 12/670,290

(22) PCT Filed: Jul. 14, 2008
(86) PCT No.: PCT/NO08/00266

§ 371 Date: Jan. 22, 2010

(30) Foreign Application Priority Data
Jul. 25, 2007 (NO) 20073911

(51) Int. Cl. A61K 31/232; A61P 9/00; A61P 25/00; A61P 19/02; A61P 37/06; A61P 17/00; A23D 7/00; C11B 5/00; A23L 1/302; A23L 1/303; A23C 9/123; A23F 5/00; A23F 3/00; A23G 1/00; A23L 2/38; A23L 2/02; A23C 9/152

(52) U.S. Cl. 514/547; 426/602; 426/541; 426/546; 426/72; 426/73; 426/583; 426/594; 426/597; 426/593; 426/590; 426/599; 426/580

(57) ABSTRACT

The present invention relates to an omega-3 fortified composition, method for its production and use of said composition as a nutritional, food or pharmaceutical composition.

### FIELD OF THE INVENTION

The present invention relates to a composition containing high amounts of omega-3 fatty acids as well as to a method for its production and the use of said composition as a nutritional, food or pharmaceutical composition.

### BACKGROUND OF THE INVENTION

Omega-3 fatty acids are considered essential fatty acids, which means that they are essential to human health but cannot be synthesised by the human body. For this reason, omega-3 fatty acids must be obtained through the diet.

The European Pharmacopoeia defines the omega-3 fatty acids as the following acids (see for example Monograph no. 1912, Fish Oil, Rich in Omega-3-Acids): alpha-linolenic acid (C18:3 n-3; ALA), moroctic acid (C18:4 n-3), eicosatetraenoic acid (C20:4 n-3), eicosapentaenoic (timnodonic) acid (C20:5 n-3; EPA), heneicosapentaenoic acid (C21:5 n-3), docosapentaenoic (clupanodonic) acid (C22:5 n-3) and docosahexaenoic (cervonic) acid (C22:6 n-3; EPA). Omega-3 fatty acids with chain-length of 20 and above are called long-chain omega-3 fatty acids. ALA is common in a number of vegetable oils. C18:4 n-3 is available from fish oils, as well as from some vegetable oils. Once eaten, the body can to some extent convert ALA and C18:4 n-3 to the long-chain omega-3 fatty acids, including EPA and DHA. However, fish oil and other marine oils are known to be the best source of these omega-3 fatty acids. Long-chain omega-3 fatty acids can also be obtained via fermentation of single cell oils (microbial oils), and research projects aim at producing EPA and DHA via gene-modified terrestrial plants.

Omega-3 fatty acids have been demonstrated to reduce the risk of coronary heart disease as well as having a positive effect on children's development, as well as on the skin. Results have also been disclosed indicating the positive effect of these fatty acids on certain mental illnesses, autoimmune diseases and joint complaints. There are therefore many reasons for considering taking fish oil as a valuable dietary supplement, including the long-term effect which this dietary supplement is now thought to have.

However, the taste and smell of fish oil are often by the consumer considered to be unpleasant. Furthermore, polyunsaturated fatty acids are highly unstable and are subjected to oxidation when exposed to air. Such processes cause the polyunsaturated fatty acids to turn rancid, giving the product an even more unpleasant smell and taste.

Some of the above-mentioned disadvantages may be overcome by preparing fish-oil fortified compositions such as fish-oil fortified milk. An emulsion of milk and fish oil is generally prepared by substituting some of the butter fat in the milk with fish oil, see e.g. WO01/80656. In low fat milk compositions, and to a large degree also in full-fat compositions, substantial amounts of the milk fat will have to be removed in order to bring in the desired amounts of long-chain omega-3 fatty acids, when fish oil are used as the omega-3 source. Thus the compositions will not have all the positive nutritional effects and taste that are often associated with diary products.

The process for preparing fish-oil fortified milk compositions is not applicable for preparing fish-oil fortified compositions based on e.g. juice or sport drinks. In order to bring in the desired amounts of long-chain omega-3 fatty acids in such products, it is often necessary to add an emulsifying agent such as lecithin.

WO2007/149590 disclose an aqueous emulsion comprising 0.65% (w/w) fish oil, wherein the fish oil contains 61% (w/w) omega-3 fatty acids and lecithin. Lecithin is most commonly used as an emulsifying agent to keep water and fats from separating in foods. Even though lecithin is regarded as a well-tolerated and non-toxic surfactant, it is also considered a possible allergen.

In EP1241955 a product is described comprising about 4% (w/w) fish oil, wherein the fish oil contains about 40% (w/w) omega-3 fatty acids, and egg yolk. Egg yolk is a source of lecithin which, as mentioned above, is considered a possible allergen.

Thus, there is a need in the art for an omega-3 fortified composition without the above-mentioned disadvantages, and a method for manufacturing such compositions.

### SUMMARY OF THE INVENTION

The object of the present invention is to provide a composition with a high content of omega-3 fatty acids but with no trace of fish oil taste or smell despite the addition of fish oil. It is also an object of the present invention that the desirable amount of omega-3 fatty acids in the composition is obtained by using as low amounts of fish oil as possible in order to avoid oily taste of the product and in order to reduce the amount of added saturated fatty acids. Furthermore, it is an object of the present invention to provide an omega-3 fortified composition with improved emulsifying properties.

The present invention has led to the novel and unexpected result of being able to substantially retain the level of diary fat, and still obtain nutritionally significant amounts of long-chain omega-3 fatty acids in omega-3 fortified milk-compositions. Thus, the composition remains surprisingly equivalent to the authentic diary product, is even though it has been fortified with long-chain omega-3 fatty acids.

Further, the omega-3 fortified composition according to the present invention which is based on low fat products, such as juice and sport drinks, has been demonstrated to have improved emulsifying properties compared with similar compositions.

Thus, a first aspect of the present invention relates to a composition comprising a liquid oil comminuted in a water-based liquid phase, wherein the liquid oil, which contains at least 600 mg/g omega-3 fatty acids, constitutes 0.05-20% (w/w) of said composition. It is preferred that said composition does not contain lecithin.

Further preferred embodiments of the first aspect of the present invention are set fourth in dependent claims 2-15.

A second aspect of the present invention relates to a method for manufacturing the composition according to the first aspect of the present invention.

A third aspect of the present invention relates to use of the composition according to the present invention as a food supplement or a nutritional, food or pharmaceutical composition.

### DESCRIPTION OF THE FIGURES

FIG. 1 Two graded cylinders, wherein cylinder A represents an emulsion of lowfat milk and fish oil A (fish oil A, see table 2) and cylinder C represents an emulsion of lowfat milk and a fish oil C (fish oil C, see example 2).

### DETAILED DESCRIPTION OF THE INVENTION

Surprisingly it has now been found that an emulsion comprising a water-based liquid, such as juice or milk, and an omega-3 fatty acid concentrate (at least 600 mg/g omega-3 fatty acids), such as EPAX 6000 TG/N (table 2), is far more stable than an emulsion comprising a water-based liquid and a fish oil containing up to about 30% (w/w) omega-3 fatty acids, such as fish oil B or C (fish oil B, see example 1; fish oil C, see example 2).

Thus, a first aspect of the present invention relates to a composition comprising a liquid oil comminuted in a water-based liquid phase, wherein the liquid oil, which contains at least 600 mg/g omega-3 fatty acids, constitutes 0.1-20% (w/w) of said composition.

Said liquid oil is preferably a marine oil or a marine-based oil (e.g. a marine-based omega-3 fatty acid concentrate) and even more preferably a fish oil or a fish-based oil (e.g. a fish-based omega-3 fatty acid concentrate, such as EPAX 6000 TG/N).

The amount of omega-3 fatty acids in said oil is at least 600 mg/g, preferably at least 650 mg/g, more preferably at least 700 mg/g and more preferably at least 800 mg/g or 900 mg/g.

The omega-3 fatty acids may exist in various forms such as fatty acids, ethyl esters, monoglycerides, diglycerides, triglycerides or phospholipids. Preferably at least 50% (w/w), 60% (w/w), 70% (w/w) or 80% (w/w) of said omega-3 fatty acids are in the form of triglycerides. More preferably at least 90% (w/w) of said omega-3 fatty acids are in the form of triglycerides, most preferably at least 95% (w/w).

Further, it is preferred that said liquid oil contains at least 70% (w/w) triglycerides, even more preferably at least 80% (w/w) triglycerides and most preferably at least 90% (w/w) or at least 95% (w/w) triglycerides.

One unit dose/serving, typically 50-200 g, of the composition according to the present invention, wherein the liquid oil constitutes 1-1.7% (w/w) of said composition, covers the recommended daily intake (RDI) of long-chain omega-3 fatty acids (0.3-2 g/day, depending on recommending authority).

However, very often one unit dose will not be intended to cover the complete RDI of long-chain omega-3 fatty acids. For example, the composition according to the present invention may contain:
a) more than 15% of the recommended nutritional intake (2 g/day) of omega-3 fatty acids per 100 g, 100 ml or 100 kcal.
b) more than 30% of the recommended nutritional intake (2 g/day) of omega-3 fatty acids per 100 g, 100 ml or 100 kcal.

The examples above are based on current EU RDI's, which include both ALA and the long-chain omega-3 fatty acids. Possible future regulations in EU of RDI for long-chain omega-3 fatty acids could be expected to be well below 2 grams.

The low amount of fat in addition to the omega-3 fatty acids will make the compositions according to the present invention well suited to comply with claims referring to percentage of RDI for omega-3 fatty acids per 100 kcal.

Thus, it is preferred that the liquid oil of the present invention contains low amounts of saturated fatty acids, preferably less than 15% (w/w), even more preferably less than 10% (w/w) and most preferably less than 8% (w/w) e.g. less than 4% (w/w) or less than 1% (w/w).

Example a) above would be fulfilled in compositions where the liquid oil constitutes 0.5% of weight, or 0.5 g/100 ml, or 0.5 g/100 kcal. Example b) would be fulfilled in compositions where the liquid oil constitutes 1% of weight, or 1 g/100 ml, or 1 g/100 kcal.

In other markets, or for other purposes, the composition could typically contain 150-250 mg long-chain omega-3 fatty acids per unit dose.

Preferably the liquid oil constitutes 0.01-20% (w/w), 0.1-15% (w/w), 0.1-10% (w/w), 0.1-8% (w/w), 0.5-15% (w/w), 0.5-10% (w/w), 0.5-8% (w/w), 2-15% (w/w), 2-10% (w/w), 2-8% (w/w), 3-7% (w/w), 4-6% (w/w), 0.5-2% (w/w) or 0.8-2% (w/w) of said composition.

EPAX 6000 TG/N is a liquid oil that contains at least 90 A % (The unit A % is defined in table 2) triglycerides and at least 65 A % omega-3 fatty acids (Table 2). Further, the oil has no fish taste or smell, and has been shown to have favourable emulsion properties (example 1-4). With that, EPAX 6000 TG/N is the preferred ingredient to be used as the liquid oil that is comminuted in the water-based liquid phase according to the present invention.

As previously mentioned, polyunsaturated fatty acids are highly unstable and are subjected to oxidation when exposed to air. These processes cause the polyunsaturated fatty acids to turn rancid, giving the product an unpleasant smell and taste. Accordingly, an oxidation preventing agent may be added to the composition according is to the present invention. Preferably, said oxidation preventing agent is selected from the group consisting of tert-butyl hydroquinone (TBHQ), butylated hydroxyanisole (BHA), butylated hydroxytoluene (BHT), one or more gallates, tocopherols, tocotrienols, acrorbic acid or ascorbic acid derivatives, natural polyphenols or polyphenol derivates, herb extracts like sage, rosemary or thyme extracts; or mixtures thereof.

“The oxidation preventing agents could also be chosen from one or more of the antioxidants tert-butyl hydroquinone (TBHQ), butylated hydroxyanisole (BHA), butylated hydroxytoluene (BHT) and/or one or more gallates.”

In order to inhibit or reduce the growth of micro organisms, a preserving agent may be added to the composition according to the present invention.

In another embodiment according to the present invention, the composition further comprises vitamins. Preferably, said vitamins are selected from the group consisting of vitamin A, B, C, D and E; derivatives and variants thereof; or mixtures thereof.

In yet another embodiment according to the present invention, the water based liquid phase is selected from the group consisting of water, mineral water, juice, milk, breast milk substitute (e.g. infant formula), yoghurt, coffee, tea, cocoa, sport drinks and sugar containing drinks, pharmaceutical product; or mixtures thereof. Preferably the water based liquid phase is juice, milk or yoghurt, and even more preferably low fat milk or a low fat yoghurt product. It should also be understood that said water based liquid phase may be a basic material that is used in the production of dairy products like e.g. cheese products.

Surprisingly, a stable emulsion of the liquid oil and the water based liquid was obtained without adding emulsifying agents (example 1-2). Accordingly, in one embodiment according to the present invention no emulsifying agents are added, and more preferably the composition of the present invention does not contain any emulsifying agents. In another embodiment, emulsifying agents are added to further stabilize the emulsion.

In one embodiment according to the present invention, the water based liquid phase is selected from the group consisting of milk, breast milk substitute, yoghurt, a basic material that is used in the production of dairy products like e.g. cheese products; or mixtures thereof. Such a composition may or may not be added an emulsifying agent, such as lecithin. However, it is preferred that no emulsifying agent is added and most preferably said composition does not contain lecithin.

In another embodiment, the water based liquid phase is selected from low fat products such as water, mineral water, juice, coffee, tea, sport drinks and sugar containing drinks; or mixtures thereof. Such a composition may or may not be added an emulsifying agent, such as lecithin. However, it is preferred that no emulsifying agent is added and most preferably said composition does not contain lecithin

One example of an emulsifying agent is lecithin. As described above, this agent is regarded as a well-tolerated and non-toxic surfactant but it is also considered a possible allergen. Accordingly, it is preferred not to add lecithin to said composition, and more preferably the composition of the present invention does not contain lecithin.

A second aspect of the present invention relates to a method for manufacturing the composition according to the present invention, wherein the method comprises the following steps:
a) heating the water based liquid phase to a temperature in the range 50° C.-90° C., preferably to a temperature in the range 60° C.-80° C., more preferably to a temperature in the range 65° C.-75° C. and most preferably to a temperature of about 70° C., e.g. 72° C.
b) adding a desired amount of liquid oil to the water based liquid phase obtained in step a);
c) cooling the mixture obtained in step b) to a temperature in the range 40° C.-60° C., preferably to a temperature in the range 45° C.-55° C., more preferably to a temperature in the range 48° C.-52° C. and most preferably to a temperature of about 50° C., e.g. 50° C.;
d) homogenizing the mixture obtained in step c)
e) optionally, adding an oxidation preventing agent and/or vitamins and/or an emulsifying agent.

Preferably, the desired amount of liquid oil that is added to the water based liquid phase to in step b) results in a mixture wherein the liquid oil constitutes 0.01-20% (w/w), 0.1-15% (w/w), 0.1-10% (w/w), 0.1-8% (w/w), 0.5-15% (w/w), 0.5-10% (w/w), 0.5-8% (w/w), 2-15% (w/w), 2-10% (w/w), 2-8% (w/w), 3-7% (w/w), 4-6% (w/w), 0.5-2% (w/w) or 0.8-2% (w/w) of said composition.

A third aspect of the present invention relates to a use of the composition according to the present invention as a nutritional, food or pharmaceutical composition.

### EXAMPLES

### Example 1

### Fish Oil A+Lowfat Milk Versus Fish Oil B+Lowfat Milk

A mixture of mini milk and lowfat milk (1:1, a total of 1% butter fat) was heated to a temperature of 72° C. Subsequently, a fish oil was added and the resultant mixture was cooled to a temperature of 50° C. The cooled mixture was then homogenized using a two-step Rannie homogenizer (pressure=50 bar, temperature 50° C., recirculation time=1 minute).

8 fish oil-milk emulsions were prepared as described above, using 2 different fish oils (fish oil A and fish oil B) at 4 different concentrations (2, 4, 6 and 8% (w/w)). The prepared fish oil-milk emulsions were then dissolved in distilled water while being vigorously stirred (2800 rpm, 14-16% obscuration). The emulsifying properties of the resultant mixture was then evaluated by visual inspection and drop size analysis (Fraunhofer method).

Visual inspection after 0, 2, 6 and 8 days.

Each of the emulsions was relatively stable (no separation) during storage irrespective of fish oil type or the concentration of the fish oil in the emulsion. However, cream formation was observed. The thickness of the creamy layer increased with the amount of oil in the emulsion. Moreover, on day 6 and 8, the emulsions containing fish oil B was observed to have a thicker creamy layer than emulsions containing fish oil A.

TABLE 1
Measurement of drop size after 2 days (the drop size after 8 days
was mainly identical to the drop size after 2 days, and is therefore
not shown).

| Fish oil | Conc. | D[3.2] | D[4.3] | D(0.1) | D(0.5) | D(0.9) |
|---|---|---|---|---|---|---|
| A | 2% | 1.44 | 1.68 | 0.92 | 1.56 | 2.61 |
| B | 2% | 1.65 | 2.10 | 0.95 | 1.91 | 3.51 |
| A | 4% | 1.50 | 1.74 | 0.97 | 1.63 | 2.65 |
| B | 4% | 1.76 | 2.13 | 1.08 | 1.99 | 3.39 |
| A | 6% | 1.45 | 1.65 | 0.95 | 1.56 | 2.49 |
| B | 6% | 1.75 | 2.09 | 1.08 | 1.95 | 3.32 |
| A | 8% | 1.42 | 1.61 | 0.94 | 1.52 | 2.41 |
| B | 8% | 1.79 | 2.13 | 1.11 | 1.98 | 3.35 |

Standard deviation <0.01 μm.
D[3.2] represents the mean surface area of the drop size calculated by using Σnᵢdᵢ³ (Σnᵢdᵢ²)⁻¹.
D[3.2] represents the mean volume of the drop size calculated by using Σnᵢdᵢ⁴ (Σnᵢdᵢ²)⁻¹.
D(0.1), d(0.5) and d(0.9) represent the drop size of the 10, 50 and 90% fractiles respectively.

TABLE 2
Fish oil A: EPAX 6000 TG/N

| | Min. | Max. | |
|---|---|---|---|
| | Value | value | Unit |
| Triglycerides* | 90 | | A %*** |
| Fatty acids and ethyl esters | | 3 | A %*** |
| Oligomers* | | 1.0 | A %*** |
| Eicosapentaenoic acid C20:5** | 34 | | A %*** |
| Docosahexaenoic acid C22:6** | 24 | | A %*** |
| Total n-3** | 65 | | A %*** |
| Total n-3: EPA, DHA, 18:3, 18:4, 20:4, 21:5, | 600 | | mg/g |
| 22:5** | | | |
| Eicosapentaenoic acid C20:5 (TG)** | 300 | | mg/g |
| Docosahexaenoic acid C22:6 (TG)* | 200 | | mg/g |
| Mixed tocopherol | 3.0 | 4.5 | mg/g |

*The contents of triglycerides (and oligomers) is (are) analysed by size-exclusion chromatography as described in the European Pharmacopoeia monograph 1352, Omega-3-acid triglycerides.
**The contents of EPA, DHA and total omega-3 fatty acids are analyzed according to the European Pharmacopoeia monograph 2.4.29, Composition of fatty acids in oils rich in omega-3-acids.
***Sum of the areas of all the peaks in the chromatogram divided by the area of the peak 10 in question (e.g. peak representing Eicosapentaenoic acid C20:5) multiplied by 100.

### Fish Oil B: Salmon Oil

The oil is produced from fresh raw materials from Salmo salar, and complies with the European Pharmacopoeia monograph no. 1910: “Salmon oil, farmed.”

### Example 2

### Fish Oil A+Lowfat Milk Versus Fish Oil C+Lowfat Milk

A mixture of lowfat milk (1.5% butter fat) and fish oil A was prepared by blending 0.2 g fish oil A per 100 ml lowfat milk for about 1 minute using a handblender. No emulsifying agents were added.

A mixture of lowfat milk (a total of 1.5% butter fat) and fish oil C was prepared by blending 0.2 g fish oil C per 100 ml lowfat milk for about 1 minute using a handblender. No emulsifying agents were added.

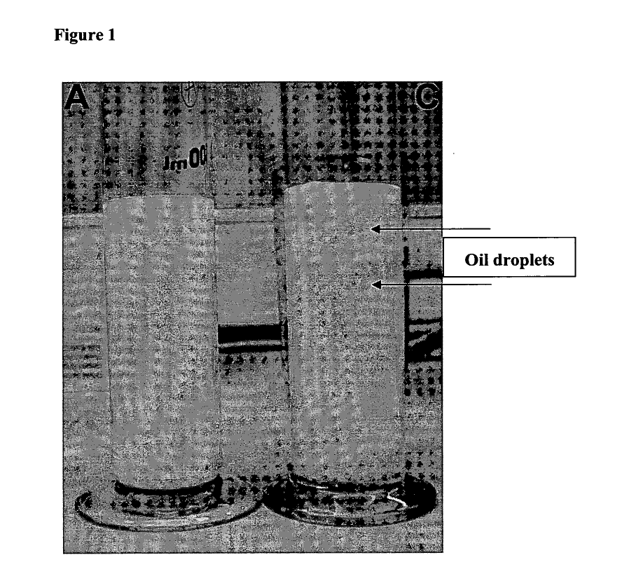

The mixtures containing fish oil A and fish oil C were then transferred to graded cylinder A (see FIG. 1, left cylinder) and graded cylinder C (see FIG. 1, right cylinder) respectively, for visual inspection.

In contrast to the mixture containing fish oil C, the mixture containing fish oil A showed no signs of drop formation (FIG. 1), which indicates that the emulsion containing fish oil A was more stable than the emulsion containing fish oil C.

### Fish Oil C

Refined oil from South America which complies with the European Pharmacopoeia monograph no. 1912: “Fish Oil, Rich in Omega-3-Acids”. The content of Omega-3 fatty acids is about 30% (w/w).

### Example 3

### Emulsifying Properties (Fish Oil A+Juice Vs Fish Oil B+Juice)

Orange juice was heated to a temperature of 30° C. Subsequently citrem was added (0.3% (w/w)) and the mixture was heated to a temperature of 70° C. Fish oil was then added and the mixture was homogenized using a two-step Rannie homogenizer (pressure=100 bar, temperature 70° C., recirculation time=1 minute).

6 fish oil-juice emulsions were prepared as described above, using 2 different fish oils (fish oil A and fish oil B) at 3 different concentrations (1, 2, and 3% (w/w)). The emulsifying properties of the resultant mixture was then evaluated by visual inspection and drop size analysis (Fraunhofer method).

Visual inspection after 4 days.

Each of the emulsions containing 1% (w/w) oil, irrespective of fish oil type, was relatively stable (no separation) during storage. However, while the emulsion containing 2% (w/w) fish oil A was relatively stable (no separation), the emulsion containing 2% (w/w) fish oil B was not stable during storage.

TABLE 3
Measurement of drop size after 7 days

| Fish oil | Conc. | D[3.2] | D[4.3] | D(0.1) | D(0.5) | D(0.9) |
|---|---|---|---|---|---|---|
| A | 1% | 0.37 | 14.203 | 0.131 | 0.789 | 52.226 |
| B | 1% | 0.425 | 18.156 | 0.138 | 1.146 | 65.801 |
| A | 2% | 0.504 | 0.504 | 0.19 | 1.162 | 22.064 |
| B | 2% | 0.536 | 12.69 | 0.183 | 1.459 | 45.329 |
| A | 3% | 0.908 | 7.656 | 0.38 | 2.782 | 11.556 |
| B | 3% | 1.013 | 8.965 | 0.483 | 3.064 | 14.421 |

D[3.2] represents the mean surface area of the drop size calculated by using Σnᵢdᵢ³ (Σnᵢdᵢ²)⁻¹.
D[4.3] represents the mean volume of the drop size calculated by using Σnᵢdᵢ⁴ (Σnᵢdᵢ²)⁻¹.
D(0.1), d(0.5) and d(0.9) represent the drop size of the 10, 50 and 90% fractiles respectively.

### Example 4

### Emulsifying Properties (Fish Oil A+Sport Drink Vs Fish Oil B+Sport Drink)

A mixture of water and maltodextrin was heated to a temperature of 35° C. Subsequently citrem was added and the mixture was heated to a temperature of 70° C. Fish oil was added and the mixture was then homogenized using a two-step Rannie homogenizer (pressure=100 bar, temperature 70° C., recirculation time=1 minute).

8 fish oil-sport drink emulsions were prepared as described above, using 2 different fish oils (fish oil A and fish oil B) at 4 different concentrations (1, 2, 3 and 4% (w/w)). The emulsifying properties of the resultant mixture was then evaluated by visual inspection and drop size analysis (Fraunhofer method).

Visual inspection after 1 day.

Each of the emulsions containing 1% (w/w) and 2% (w/w) oil, irrespective of fish oil type, were relatively stable (no separation) during storage. However, while the emulsions containing 3% (w/w) or 4% (w/w) fish oil A were relatively stable (no separation), the emulsions containing 3% (w/w) or 4% (w/w) fish oil B were not stable during storage.

TABLE 4
Measurement of drop size after 7 days

| Fish oil | Conc. | D[3.2] | D[4.3] | D(0.1) | D(0.5) | D(0.9) |
|---|---|---|---|---|---|---|
| A | 1% | 0.256 | 0.511 | 0.111 | 0.435 | 1.032 |
| B | 1% | 0.323 | 0.705 | 0.131 | 0.634 | 1.391 |
| A | 2% | 0.322 | 2.589 | 0.136 | 0.539 | 5.544 |
| B | 2% | 0.342 | 0.709 | 0.144 | 0.637 | 1.381 |
| A | 3% | 0.31 | 0.606 | 0.134 | 0.536 | 1.188 |
| B | 3% | 0.509 | 5.45 | 0.236 | 0.826 | 19.132 |
| A | 4% | 0.445 | 3.539 | 0.212 | 0.659 | 2.075 |
| B | 4% | 0.586 | 5.947 | 0.269 | 0.934 | 21.692 |

D[3.2] represents the mean surface area of the drop size calculated by using Σnᵢdᵢ³ (Σnᵢdᵢ²)⁻¹.
D[4.3] represents the mean volume of the drop size calculated by using Σnᵢdᵢ⁴ (Σnᵢdᵢ²)⁻¹.
D(0.1), d(0.5) and d(0.9) represent the drop size of the 10, 50 and 90% fractiles respectively.

1. A composition comprising a marine oil or marine-based oil comminuted in a water-based liquid phase, wherein the marine oil or marine-based oil, which contains at least 600 mg/g omega-3 fatty acids and at least 70% (w/w) triglycerides, constitutes 0.05-20% (w/w) of said composition, with the proviso that said composition does not contain lecithin.

2. The composition according to claim 1, wherein the marine oil or marine-based oil constitutes 0.1-10% (w/w) of said composition.

3. The composition according to claim 2, wherein the marine oil or marine-based oil constitutes 0.5-2% (w/w) of said composition.

4. The composition according to claim 1, wherein at least 80% (w/w) of said omega-3 fatty acids are in the form of triglycerides.

5. The composition according to claim 1, wherein the marine oil or marine-based oil contains at least 90% (w/w) triglycerides.

6. The composition according to claim 1, wherein the marine oil or marine-based oil contains at least 95% (w/w) triglycerides.

7. The composition according to claim 1, wherein the amount of saturated fatty acids in said marine oil or marine-based oil is no more than 8% (w/w).

8. The composition according to claim 1, wherein the liquid marine oil or marine-based oil is EPAX 6000 TG/N.

9. The composition according to claim 1, further comprising an oxidation preventing agent(s).

10. The composition according to claim 9, wherein the oxidation preventing agent(s) is selected from the group consisting tert-butyl hydroquinone (TBHQ), butylated hydroxyanisole (BHA), butylated hydroxytoluene (BHT), one or more gallates, tocopherols, tocotrienols, acrorbic acid or ascorbic acid derivatives, natural polyphenols or polyphenol derivates, herb extracts like sage, rosemary or thyme extracts; or mixtures thereof.

11. The composition according to claim 1, further comprising vitamin(s).

12. The composition according to claim 1, further comprising vitamin(s) selected from the group consisting of vitamin A, B, C, D and E; or mixtures thereof.

13. The composition according to claim 1, wherein the water based liquid phase is selected from the group consisting of water, mineral water, juice, milk, breast milk substitute, yoghurt, coffee, tea, cocoa, sport drinks and sugar containing drinks, and a basic material that is used in the production of dairy products.

14. The composition according to claim 1, wherein the water based liquid phase is selected from the group consisting of milk, breast milk substitute, yoghurt, and a basic material that is used in the production of dairy products.

15. (canceled)

16. A method for manufacturing the composition according to claim 1, the method comprising the following steps:
a) heating the water based liquid phase to a temperature in the range 50° C.-90° C.;
b) adding a desired amount of marine oil or marine-based oil to the water based liquid phase obtained in step a);
c) cooling the mixture obtained in step b) to a temperature in the range 40° C.-60° C.; and
d) homogenizing the mixture obtained in step c).

17. (canceled)
